(19) United States
(12) Patent Application Publication
(10) Pub. No.: US 2022/0211877 A1
(43) Pub. Date: Jul. 7, 2022

(54) COMPOUND FOR INTRAOPERATIVE MOLECULAR BIOIMAGING, METHOD OF MAKING THE SAME, USE THEREOF IN INTRAOPERATIVE MOLECULAR BIOIMAGING AND SURGICAL METHOD COMPRISING INTRAOPERATIVE MOLECULAR BIOIMAGING

(71) Applicants: Klinikum rechts der lsar der Techischen Universität München, München (DE); Techischen Universitat Munchen, Munich (DE)

(72) Inventors: Markus Nieberler, München (DE); Florian Reichart, Munich (DE); Horst Kessler, Schwalbach a. T. (DE)

(21) Appl. No.: 16/967,624

(22) PCT Filed: Feb. 6, 2019
(86) PCT No.: PCT/EP2019/052883

§ 371 Date: Aug. 5, 2020

(30) Foreign Application Priority Data
Feb. 6, 2018 (EP) 18155264.7

(51) Int. Cl. A61K 49/00; A61P 35/00; A61P 41/00

(52) U.S. Cl. CPC A61K 49/0032; A61P 41/00; A61P 35/00; A61K 49/0056

(57) ABSTRACT

The present invention provides compounds suitable for intraoperative bioimaging. In particular, compounds are provided that are suitable for marking tumor tissue during surgery to facilitate complete removal of said tumor tissue. Corresponding methods for intraoperative bioimaging and methods for surgery comprising intraoperative bioimaging are also provided.

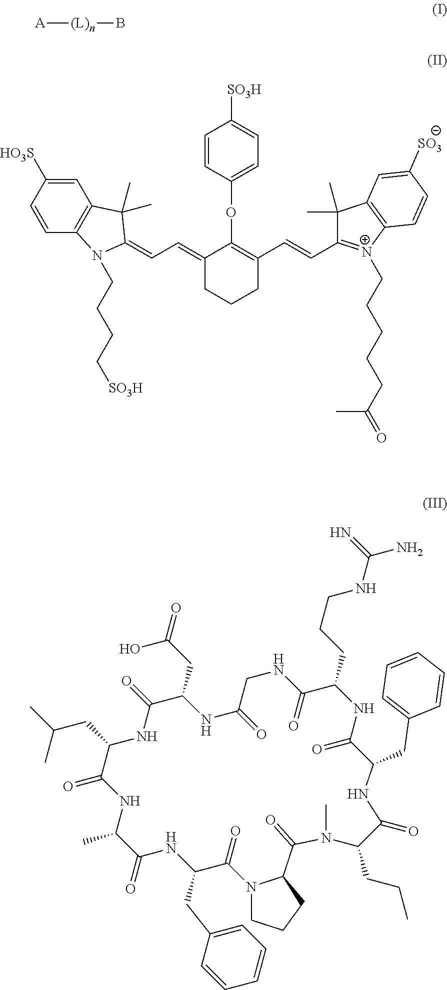

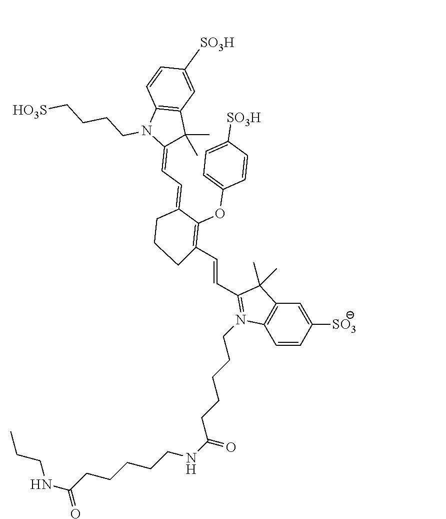

### CROSS REFERENCE TO RELATED APPLICATIONS(S)

This Application is a National stage filing under 35 U.S.C. 371 of International Application No. PCT/EP2019/052883, filed Feb. 6, 2019, which claims priority to European Application No. 18155264.7, filed Feb. 6, 2018, each of which is herein incorporated by reference in their entirety.

### FIELD OF THE INVENTION

The present invention provides compounds suitable for intraoperative bioimaging. In particular, compounds are provided that are suitable for marking tumor tissue during surgery to facilitate complete removal of said tumor tissue. Corresponding methods for intraoperative bioimaging and methods for surgery comprising intraoperative bioimaging are also provided.

### BACKGROUND OF THE INVENTION

The constant development of data acquisition, storage and processing enables medical methods and therapeutic strategies to be increasingly customized for patients. As part of the pretherapeutic staging procedure, oncological relevant information about the localization, the tissue density, the tumour extent, perfusion and/or the metabolic status and the degree of differentiation of the primary tumour and potential filiae are obtained. The amount of information makes it possible to develop a guideline-oriented, personalized therapy concept for each patient based on an interdisciplinary consensus. For a primarily surgical therapy concept with curative intention, the information of the staging procedure cannot be applied without loss of information for intraoperative detection and demarcation of malignant tissue. The intraoperative acquisition of information on the extent and localization of invasive carcinoma tissue are limited to the clinical-visual and palpatory findings and intraoperative histological control is limited to representative areas of the soft tissue resection margins. Loci-regional recurrences and metastases can result from potential residues of invasive carcinoma cells and represent limiting factors in curative therapy concepts.

In particular, an incomplete removal of malignant tissue reduces the success of oncological therapy concepts. In head and neck cancers for example, residual carcinoma tissue (R1 status) results in a 100% increase of local recurrence, with a 90% risk of death at 5 years. ^{1,2}

If microscopic fate of malignant tissue is detected during histological examination following tumour surgery, complete removal may be attempted in a second operation, and/or the therapeutic concept may be adjuvant, i.e. postoperative treatment by radiation and/or chemotherapy. Despite the option of resection in a second surgical procedure and advances in adjuvant therapy concepts, incomplete removal of malignant tumours is often associated with higher metastasis, greater recurrence rate, higher morbidity, worse prognosis, and higher costs. ^{1,3} The limitations resulting from the lack of intraoperative diagnosis are particularly evident in disciplines where surgical procedures are performed in tight anatomical conditions with functionally important tissue structures. In the case of head and neck carcinomas in particular, the incomplete removal of a malignant tumour is the greatest risk factor for dying from the disease. ⁴Thus, despite major advances in imaging techniques, reconstructive surgical techniques, and adjuvant therapy concepts, the lacking control over invasive carcinoma cells at the resection margin remains the major risk factor.

There have been efforts to solve this problem by means of fluorescence guided surgery. ^{8, 9}

However, all approaches so far are either based on αvβ3-targeting strategies or on established antibodies, conjugated with fluorescent dyes.

Based on the hypothesis that epithelial cells, which proliferate, dedifferentiate, and infiltrate as invasive carcinoma cells into mesenchymal tissue, undergo molecular and morphological epithelial-mesenchymal transition (EMT)-associated changes, the EMT-markerprotein integrin αvβ6 is considered as an appropriate target for invasive carcinoma cells. The EMT is a crucial physiological process during embryonic development. Corresponding cellular alterations have also been observed during fibrotic tissue remodeling, wound healing, invasive carcinoma growth, and tumor metastasis. ¹⁰ In an oncologic context, the membrane-bound expression of integrin αvβ6 mediates invasive carcinoma cells oncogenic potential, inhibits apoptosis and promotes invasive proliferation, as shown in FIG. 1. The expression of αvβ6 is initiated during embryonic development with high levels exclusively restricted to epithelial cells, the developing lung tissue, and the kidney epithelia. ^{11,12} In a physiological context, αvβ6 is not constitutively expressed in differentiated epithelial cells. However, it becomes upregulated again in the context of tissue remodeling, including wound healing and carcinogenesis. ¹³ The prevalence of αvβ6 expression has been described in several different kinds of malignancies. αvβ6 is referred to as “the cancer integrin”. ¹⁴ αvβ6 expression is known in the literature to be associated with invasive cancer and metastasis. ¹⁵⁻³⁸

Compounds selectively binding to αvβ6 are known. ^{40, 40a} It has also been suggested to couple such compounds to labelling substances for imaging purposes. However, the specific use in intraoperative imaging is not mentioned.

There is also a publication on intraoperative bioimaging using another compound. ^{40b} However, while the authors describe said compound to exhibit αvβ6 specific binding, there are concerns that said compound may also bind to αvβ3, so that the suitability of said compound for intraoperative bioimaging of tumor tissue of head and neck cancers, which do express αvβ6, remains questionable.

Coupling a dye to a peptide ligand involves a risk of negatively influencing binding characteristics of the ligand. Moreover, the dye may influence bioavailability and distribution within the body. These are also important properties for successful intraoperative bioimaging.

### Object of the Present Invention

In view of the above difficulties, it is an object of the present invention to provide compounds that are suitable for intraoperative bioimaging. In particular, it is an object of the present invention to provide compounds containing a peptide ligand moiety and a fluorescent dye moiety, which exhibit a high affinity and selectivity for αvβ6-expressing cancer cells, a high tissue to background ratio of fluorescence intensity and which exhibit high bioavailability and do not accumulate in healthy tissue. According to further objects of the invention, such compounds are to be provided, which additionally exhibit high biological stability and long fluorescence stability.

Further objects of the present invention pertain to the provision of methods for manufacturing said compounds as well as uses and methods of said compounds for intraoperative bioimaging.

### SUMMARY OF THE PRESENT INVENTION

The present invention solves the above problem by providing compounds that accomplish the above objectives and thus allow the intraoperative molecular bioimaging of invasive carcinoma cells to obtain direct visual information about the location and extent of invasive carcinoma tissue in real-time. The intraoperative visual detection and demarcation of carcinoma tissue can assist surgical treatment to improve the control of invasive carcinoma cells at the resection margin and contribute to surgical therapy concepts with curative intention.

In particular, the present invention provides compounds suitable for intraoperative bioimaging as specified in appended claim 1.

Preferred embodiments of the compounds of the present invention are specified in appended dependent claims 2 to 4.

The invention further provides methods for making the compounds of the present invention, as specified in appended claim 5.

Finally, the present invention also provides uses of the compounds of the present invention in intraoperative bioimaging as specified in appended claims 6 to 8.

### DESCRIPTION OF FIGURES

FIG. 1: Integrin functions instrumental for tumor biologically relevant processes:

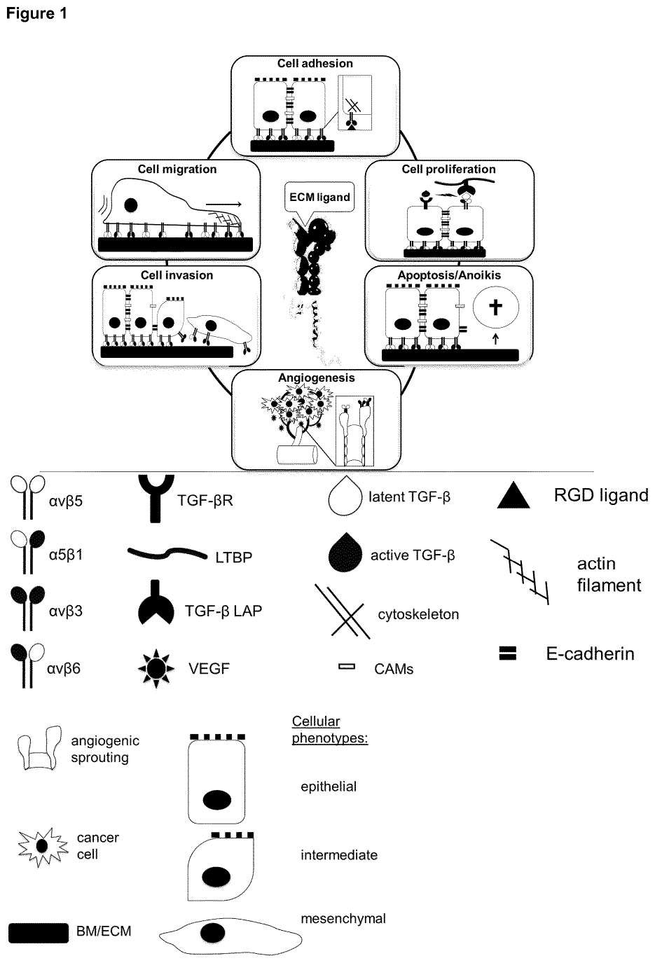

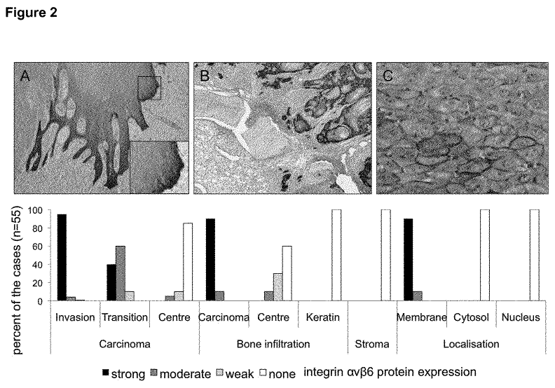

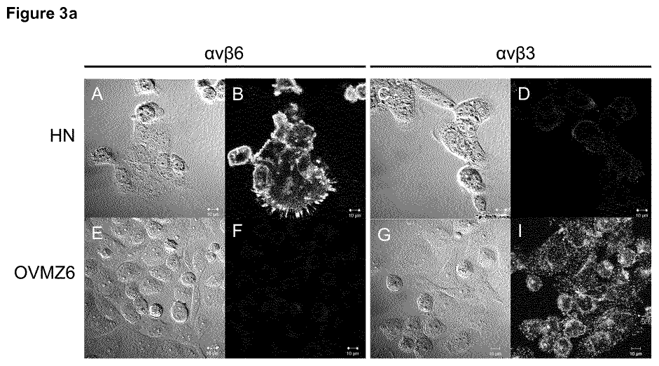

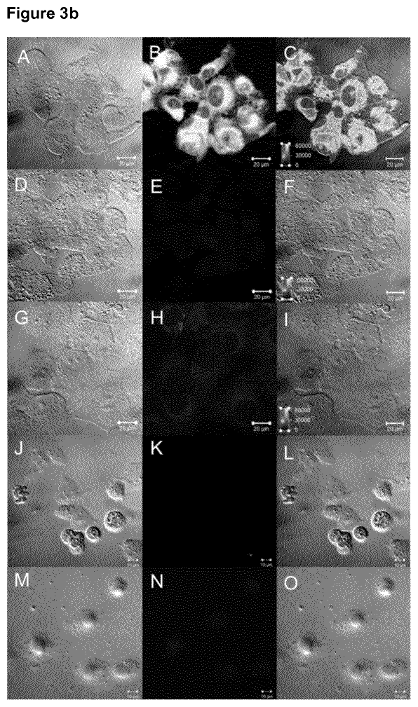

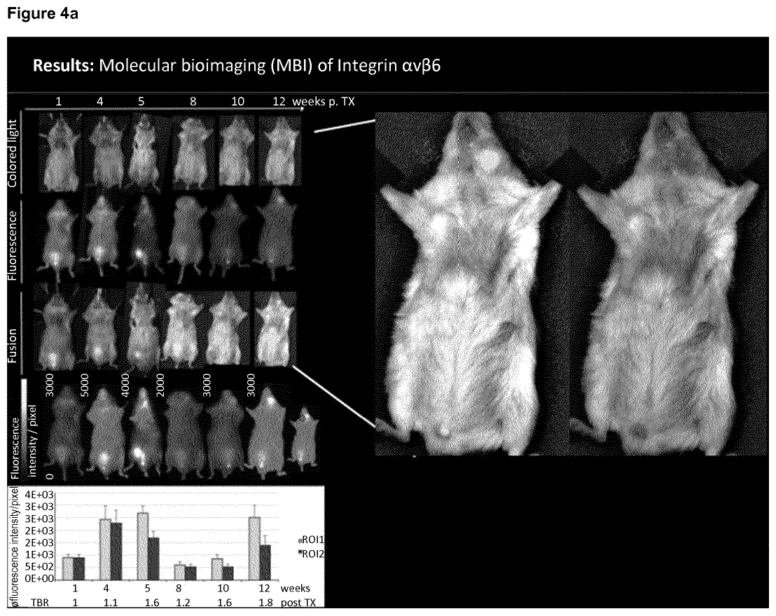

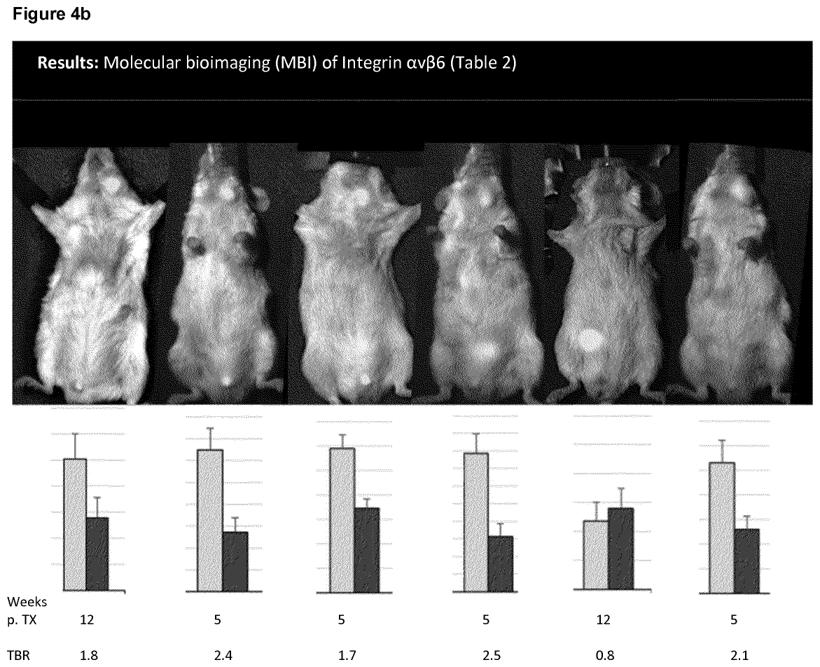

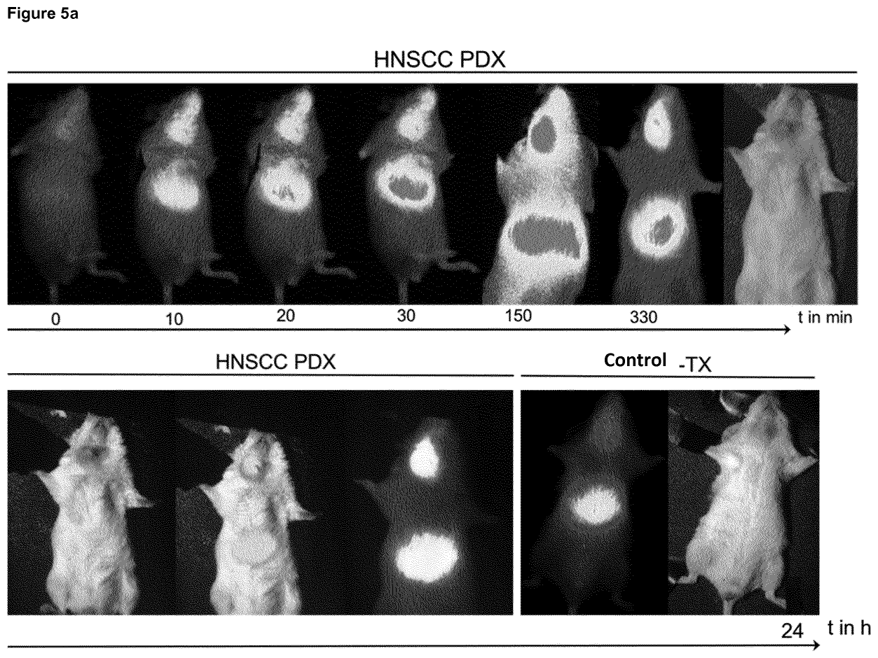

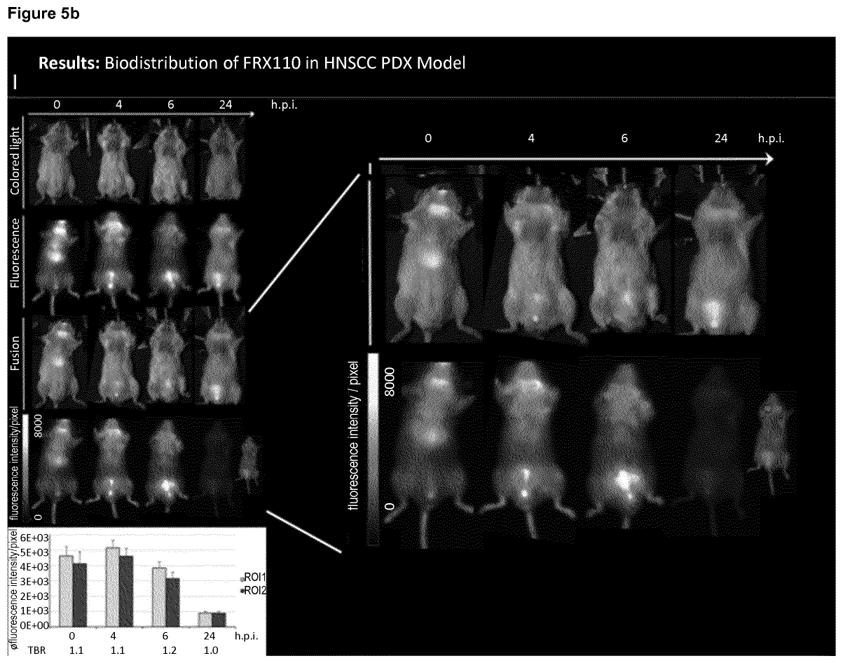

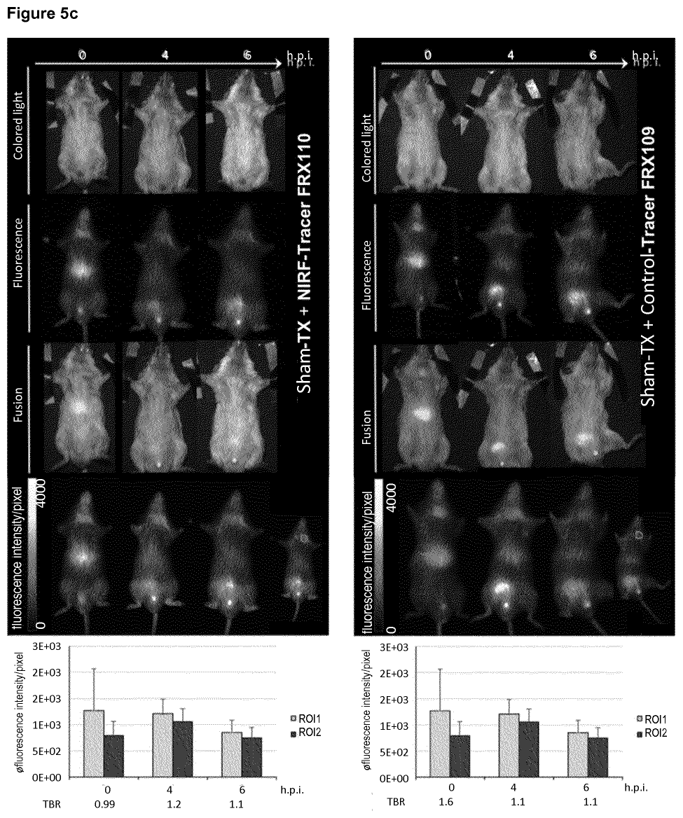

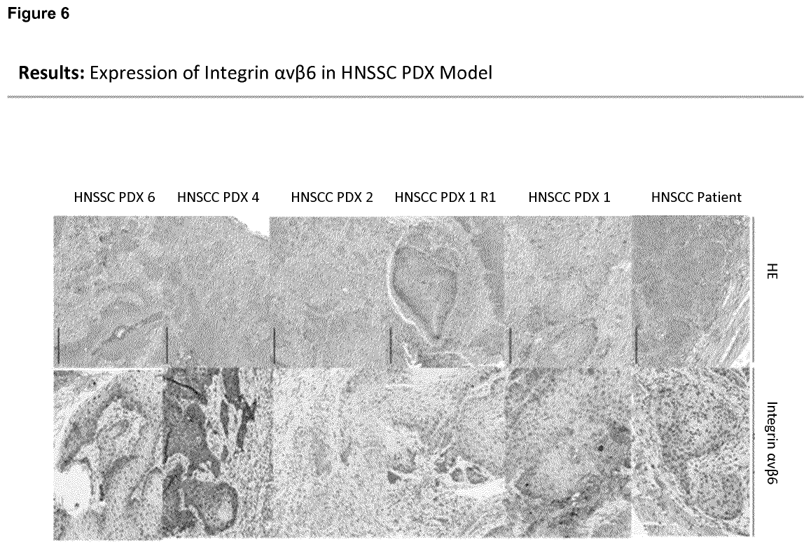

Integrin functions are involved in tumor biological processes, including cell adhesion, proliferation, inhibition of apoptosis/anoikis, induction of angiogenesis, as well as cell invasion and migration.

Cell adhesion is mediated by binding of integrins to the respective recognition motif in ECM ligands, e.g. fibronectin, osteopontin or vitronectin.

Cell proliferation mediated by integrin subtypes, such as αvβ6 and αvβ8, may also be induced upon binding of the RGD-containing latency associated (LAP) of the inactive TGF-β molecule, resulting in the activation of the latent TGF-β molecule. Subsequent binding of TGF-β to TGF-β receptors induces epithelial-mesenchymal transition (EMT) and cell proliferation.

Integrin expression allows cells to bind to ECM molecules in a mesenchymal tissue context, thereby inhibiting apoptosis/anoikis of invasive carcinoma cells. Furthermore, it induces vessel sprouting and angiogenesis. The integrin switch αvβ5 to αvβ6 allows cells to migrate and to invade into surrounding tissue.

FIG. 2: Immunohistochemical illustration of integrin αvβ6 expression in invasive OSCCs

FIG. 3a: Example 3.1 Immunofluorescence cytological expression analysis of integrin αvβ6 and αvβ3 in HN and OVMZ6 cell, respectively.

FIG. 3b: In vitro bioimaging of carcinoma cells and keratinocytes with integrin αvβ6 selective RGD-peptides and the non-integrin binding control peptide.

FIG. 4a, 4b: Intraoperative bioimaging (visualization) of invasive carcinoma tissue using the integrin αvβ6-specific, NIRF-functionalized RGD peptide FRX110 in HNSCC PDX model

FIG. 5a: Molecular bioimaging of integrin αvβ6 expression using NIRF tracer FRX103 in the HNSCC PDX model and in control animals.

FIG. 5b: Molecular bioimaging and biodistribution of NIRF tracer FRX110

FIG. 5c: FRX110 and FRX109 in control animals with sham transplantation

FIG. 6: Histologic/Immunohistologic representations of tissue excised in molecular bioimaging assisted surgery of inventive example 2

### DETAILED DESCRIPTION

### Definitions

In the context of the present invention, the terms “linker” and “spacer” are used synonymously to characterize a divalent chemical group that connects the peptide ligand moiety A with the dye moiety B such that the distance between the two moieties is increased by at least the length of two covalent bonds. References to “linker” should be understood as references to “spacer” and vice versa.

“Precursor” refers to a compound that carries a functional group and that yields the respective moiety upon one or more chemical reaction steps, typically coupling steps. Of course the precursor may also carry protective groups as appropriate in view of the contemplated synthetic route.

For instance, a precursor for the peptide moiety A may be the peptide itself or a modified form thereof, into which reactive groups and/or protective groups have been incorporated as required.

“Functional group” or “reactive functional group” are used synonymously to characterize a group that is capable of undergoing the desired reaction. Said group may be activated as appropriate in view of the desired reaction.

Indications of compounds of the present invention in specific salt forms or free acid forms or free base forms are provided for illustrative purposes and are not meant to be limiting the scope of the respective compounds to the depicted form. In other words, it should be understood that indications of the compounds of the present invention are intended to characterize the respective compounds in any form including the free acid form, free base form and salt form with any pharmaceutically acceptable counter-ion. Considering that the compounds of the present invention have multiple acidic and basic functional groups, the above statement applies independently to each of these ionizable functional groups.

Moiety a that Selectively Binds to αvβ6

In principle, any moiety may be used, which exhibits a strong, selective binding to αvβ6. The present invention specifically provides compounds, wherein the moiety A is derived from compound 18 of WO 2017/046416 A, which is a cyclic nonapeptide cyc(FRGDLAFp(NMe)K). This peptide compound has been shown in WO 2017/046416 A to exhibit high binding affinity and selectivity to αvβ6. In the examples section, it is referred to as compound OM_1204.

Moiety B that is Derived from Fluorescent Dye

The moiety B is derived from a fluorescent dye commercially available from Li-Cor® as IRDye® 800CW. Further information on this dye can be obtained from the manufacturer under licor.com/bio/products/reagents/irdye/800cw/index.html.

The structure of this dye is shown below (in the form of the reactive NHS-ester):

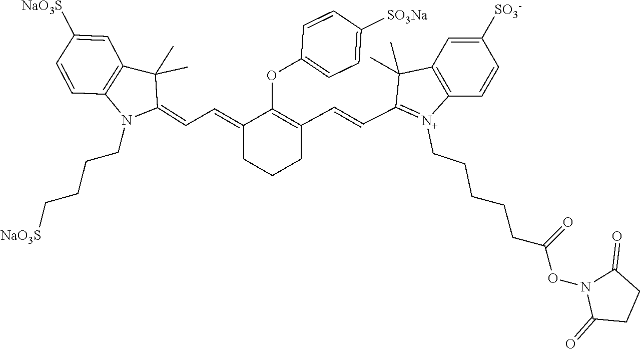

This dye is also available in the form of a reactive maleimide, which has the following structure:

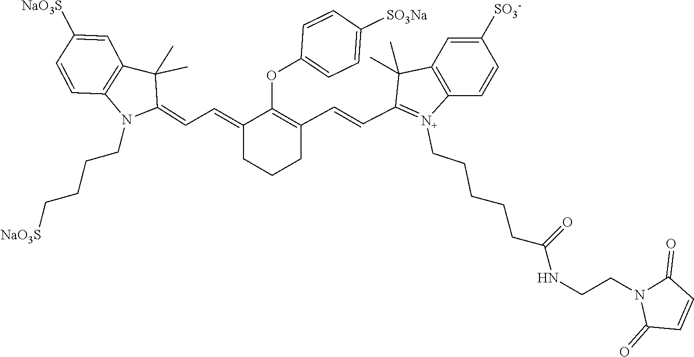

Further versions of the dye are available, which carry a carboxylate group or azide group or alkyne group or dibenzocyclooctyne (DBCO) as respective functional groups. The coupling partner and coupling reaction conditions must be suitably matched with the selected version of the dye. For instance, the DBCO version of the dye is suitable for Cu-free click chemistry coupling with an azide functional group by means of a strain-promoted alkyne azide cycloaddition, which means that the coupling partner must be selected to have an azide group.

In the present invention, it is preferred to use the NHS-ester compound as a starting material. When coupled with an amino group of the peptide or linker, the NHS-ester group will be replaced by said amino group to yield an amide group.

Linker/Spacer

The linker connects the peptide moiety A with the dye moiety B. Employing a linker with a suitable length can be advantageous as a means for minimizing interference of the peptide moiety binding to the αvβ6 by the dye moiety. In this manner, the affinity and selectivity of the peptide binding can be retained and possibly even increased.

In principle any divalent chemical group may be used in the context of the present invention as a linker. Suitable are in particular the linkers described in WO 2017/046416 A. Preferred are linkers that are derived from precursor compounds having two functional groups that are reactive towards the functional groups of the bonding partners. Such functional groups can be (activated or non-activated) carboxylic acid groups, amino groups, functional groups suitable for click chemistry couplings such as azide groups, ethinyl groups and dibenzocyclooctyne (DBCO) groups.

For instance, the ω-amino group of the lysine residue is advantageously reacted with a carboxyl group to generate an amide group.

The NHS-ester group of the IRDye® 800 CW may be coupled with an amino group, also generating an amide group.

To make use of these favourable coupling reactions, it is preferred to employ a linker, which carries a carboxyl group at one terminus and an amino group at the other terminus. These functional groups may in the course of the synthesis be suitably protected, as required.

Between the two reactive groups, the linker typically contains a chain of atoms. The length of this chain is preferably from 1 to 10 atoms in the backbone of the linker. These atoms are typically independently selected from C, O, N, S and P, with C being preferred. Free valencies of these atoms are of course saturated by hydrogen or non-reactive substituent groups that do not interfere with the binding of the peptide moiety to the αvβ6 target receptor. More preferred are linkers having an alkylene chain of 4-6 carbon atoms. Preferred are linkers derived from w-amino alkyl carboxylic acids with 5 to 7 carbon atoms. Most preferred is the use of a linker derived from 6-aminohexanoic acid (AHX).

Compound of the Present Invention and Method of Manufacture

The compounds of the present invention are obtained from compound 18 of WO 2017/046416 A and the fluorescent dye by means of a coupling reaction. Optionally and preferably, this coupling is accomplished via a spacer/linker. By consequence, the compounds of the present invention may be characterized by the following general formula:

A-(L)ₙ-B

wherein A refers to the moiety derived from compound 18 of WO 2017/046416 A, L is the linker, n is 0 or 1 and B is the moiety derived from the fluorescent dye.

The coupling position in the peptide moiety is the ω-amino group of the lysine residue. Coupling via substituents introduced into the proline sidechain or the sidechain of the neighbouring phenylalanine may, in principle be also possible, but greater synthetic efforts would be needed and at least for this reason, this option is not preferred.

As noted above, the use of an extra linker/spacer is not strictly necessary. This is because the reactive NHS-ester coupling group of the IRDye® 800 CW is separated from the chromophore by an alkylene chain of five carbon atoms. This alkylene chain (together with the lysine side chain) may function as an “internal” linker/spacer, accomplishing the desired spatial separation of the peptide moiety A from the dye moiety B.

A preferred compound of the present invention is derived from compound 18 of WO 2017/046416 A, IRDye® 800 CW NHS-ester and an 6-aminohexanoic (Ahx) spacer. A particularly preferred compound has the following structure (wherein the moiety derived from the dye is highlighted):

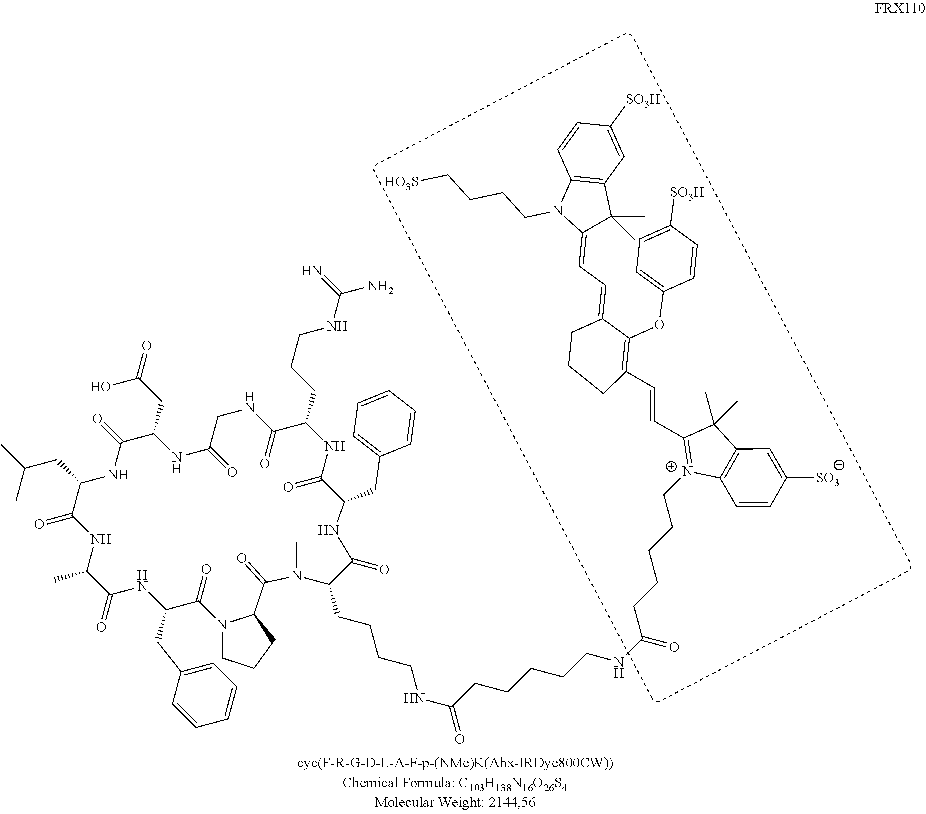

The compounds of the present invention may be prepared using conventional coupling reactions between suitable reactive groups. The sequence of the individual steps is not restricted. It thus involves the following steps in any reasonable order:
Providing the peptide moiety precursor;
Providing the precursor for the linker;
Coupling one functional group of the peptide moiety precursor with the linker precursor;
Providing the dye precursor;
Coupling the other functional group of the linker precursor with the dye precursor.

The peptide moiety precursor may be provided relying on the synthetic methods described in WO 2017/046416 A. The dye precursor may be provided relying on commercial sources such as the supplier Li-Cor®. The linker precursor may also be commercially available or be synthesized using procedures well-known in the art.

Coupling reactions are performed relying on established procedures well-known in the art.

According to one aspect, the coupling between peptide moiety A and linker may be done first, followed by coupling of the resulting molecule to the dye precursor. According to another aspect, the linker precursor may first be coupled to the dye precursor, followed by coupling of the resulting molecule to the peptide moiety precursor. According to yet another aspect, the coupling of the linker precursor to the peptide moiety precursor may be integrated into peptide synthesis. For instance, the linker may be coupled with a linear peptide precursor. After this coupling step, the peptide is cyclized, followed by coupling with the dye precursor. This last alternative is illustrated by the procedure of Example 1 below.

Use of Compound of the Present Invention

The compounds of the present invention may be used for in vivo marking and detection of invasive carcinoma tissue. This characteristic qualifies the compounds of the present invention for use in intraoperative bioimaging.

Specific uses of the compounds of the present invention include also preoperative demarcation, intraoperative demarcation and control of the resection side to control for residual invasive αvβ6 positive carcinoma cells and postoperative control of the resection side, which can also be applied as an addition means for the aftercare of tumor patients. All αvβ6 positive malignancies that can be considered for the above mentioned applications are summarized in Table 1 and marked in green.

Further possible uses in this context are control of resection status and mapping of lymph nodes.

Intraoperative bioimaging is known in the art. Pertinent literature has been reviewed recently.⁴¹ Intraoperative bioimaging may be carried out as described in this review article and literature cited therein (of course with the main difference that a compound according to the present invention is used instead of the compounds described in this article). References ⁴² and ⁴³ also describe suitable methods for intraoperative bioimaging, which may be adapted to the compounds of the present invention.

In principle, intraoperative bioimaging involves the following steps/actions:
(a) Systemic administration of the compound. This is typically done by i.v. administration although other administration forms are not per se excluded (as long as the required biodistribution and stability are accomplished).
   There is no particular limitation on the formulation to be administered. For i.v. administration, typical solutions/suspensions for injection may be used. Particularly suitable is a formulation based on sterile phosphate buffered saline (PBS) with 5% dimethylsulfoxid (DMSO). Total dose and concentration are suitably adjusted such that the desired TBR (tumor to background ratio in fluorescence intensity) is accomplished. The time interval between administration and surgery is also suitably adjusted to accomplish a desired TBR. A time interval of 24 h may give satisfactory results.
(b) Irradiating the tissue with suspected tumor cells with light capable of inducing fluorescence emission by the dye moiety. This is advantageously done by means of a laser. Suitable equipment for irradiation is described in literature ⁴² and especially the laser light source described in connection with the first camera system described in Section “2.2 Imaging Systems” thereof.
(c) Detecting fluorescence emission. Again, suitable equipment is described in literature ⁴² and especially the first camera system described in Section “2.2 Imaging Systems” thereof.
(d) Surgically removing tissue that exhibits fluorescence emission or that is surrounded by tissue exhibiting fluorescence emission.

The compounds of the present invention may be used for intraoperative bioimaging and related uses in connection with any type of cancer, wherein αvβ6 is expressed. This includes in particular colon cancer, gastric carcinoma, oral squamous cell carcinoma (OSCC), pancreatic ductal adenocarcinoma, intestinal adenocarcinoma, head and neck squamous cell carcinoma (HNSCC), invasive endometrial carcinoma, basal cell carcinoma, breast cancer, endometrial cancer, gastric cancer, liver cancer, non small cell lung cancer, lung cancer brain metastases, ovarian cancer, pancreatic cancer and prostate cancer.

### EXAMPLES

### 1. Synthesis Example—Synthesis of Compounds of Present Invention and of Comparative Compounds

1.1 Synthesis of Cyclic Peptides Carrying a Linker: Cyc(FRGDLAFp(NMe)K(Ahx)) as a Precursor of a Compound of the Present Invention and Cyc(FRADLAFp(NMe)K(Ahx)) as a Precursor of a Comparative Compound

The cyclic peptides were synthesized on a 2-chloro-tritylchloride polystyrene (2-CTC) resin following standard Fmoc strategy⁴⁶ and subsequent cyclization in solution. N-Methylation was performed on resin as described elsewhere.⁴⁷ In brief, Fmoc protected glycine (or alanine, resp.) (1.2 eq) was immobilized on the resin (0.969 mmol/g) with DIEA (2.5 eq) in anhydrous DCM (2 mL) for 1 h. Elongation of the peptide chain was done using Fmoc-Xaa-OH (2 eq), HATU (2 eq), HOBt (2 eq) and DIEA (5 eq) in DMF for 1 h. Dde protection of the side-chain of lysine was used for the orthogonal on-resin modification of the linear peptides with Boc-Ahx-OH. After cleavage from the resin with 20% HFIP in DCM, the linear peptides were cyclized using DPPA (3 eq) and NaHCO₃ (5 eq) in DMF for 16 h. The cyclic peptides were subsequently deprotected with TFA/DCM/TIPS/water (80:15:2.5:2.5%) for 1 h.

1.2 Coupling of Peptide-Linker Precursors with Dye Precursors

The fluorescent labelling was performed using HPLC purified cyc(FRGDLAFp(NMe)K(Ahx)) or cyc(FRADLAFp(NMe)K(Ahx)), resp. (1 eq), Cyanine-5.5 NHS ester (1 eq), or Cyanine-7.5 NHS ester, or IRDye800CW NHS-Ester) and DIEA (3 eq) in DMF for 1 h (monitoring by HPLC-MS). The conjugates were finally purified by semi-preparative HPLC and lyophilized to yield the target compounds OM1231, FRX103, FRX109 or FRX110, respectively.

Compound OM1231 is a comparative compound containing a peptide moiety derived from compound 18 of WO 2017/046416 A, which is coupled to a dye moiety derived from the Cyanine-5.5 dye via the AHX linker.

Compound FRX103 is another comparative compound that corresponds to OM1231, but wherein the Cyanine-7.5 dye is incorporated instead of the Cyanine-5.5 dye.

The structure of OM1231 as well as the differing dye moiety of FRX103 are shown below:

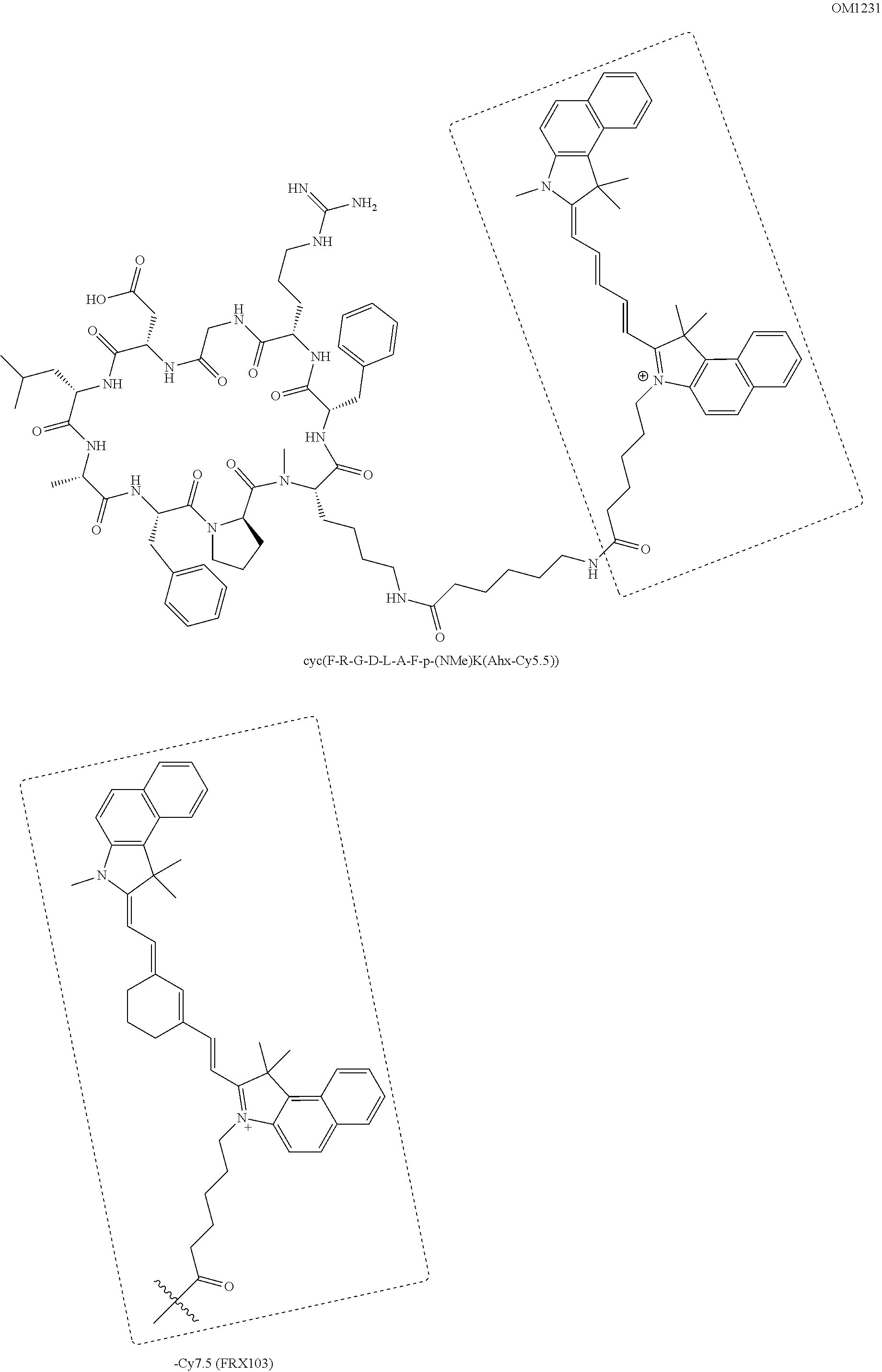 [SMILES: CC(C)C[C@@H]1NC(=O)[C@H](CC(=O)O)NC(=O)CNC(=O)[C@H](CCCNC(=N)N)NC(=O)[C@H](Cc2ccccc2)NC(=O)[C@H](CCCCNC(=O)CCCCCNC(=O)CCCCC[N+]2=C(/C=C/C=C/C=C3/N(C)c4ccc5ccccc5c4C3(C)C)C(C)(C)c3c2ccc2ccccc32)N(C)C(=O)[C@H]2CCCN2C(=O)[C@H](Cc2ccccc2)NC(=O)[C@H](C)NC1=O ; *C(=O)CCCCC[N+]1=C(/C=C/C2=CC(=C/C=C3/N(C)c4ccc5ccccc5c4C3(C)C)/CCC2)C(C)(C)c2c1ccc1ccccc21]

Compound FRX110 is a compound of the present invention, which has the following structure:

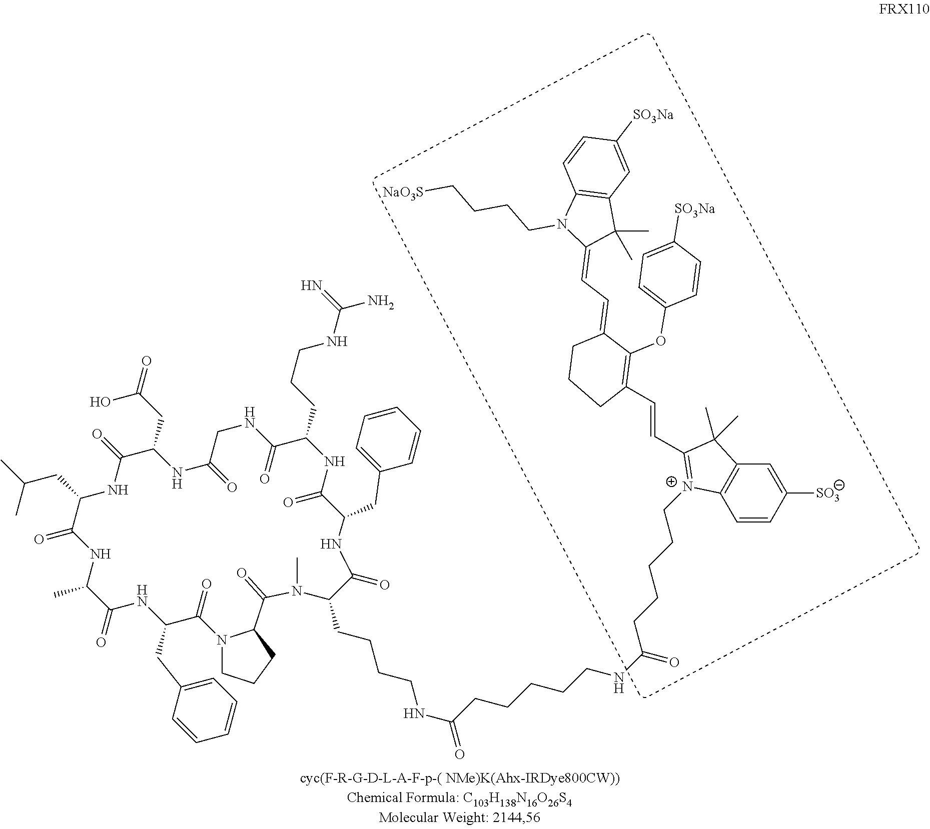

Compound FRX109 is a comparative compound, wherein the alternative peptide-linker moiety cyc(FRADLAFp(NMe)K(Ahx)) is coupled to the IRDye 800CW-derived moiety. It has the following structure:

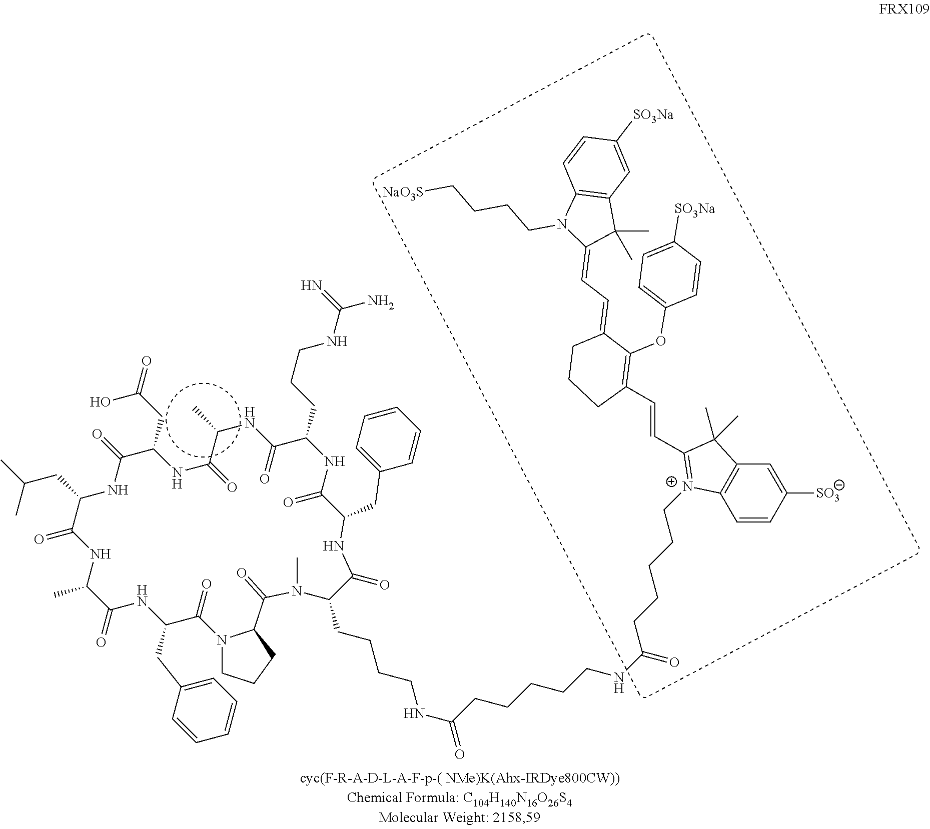

The integrin binding affinity and selectivity profile of the compounds was determined by a solid-phase binding assay as previously described.⁴⁸

The results of these determinations are summarized in Table 1 below. To allow better comparison, the binding profile of the parent compound (i.e. compound 18 of WO 2017/046416 A, designated as compound OM1204) is also included in the table.

For ease of reference, the relationship between the employed compound codes and sequences is summarized below:

| code | sequence |
|---|---|
| OM1204 | cyc(FRGDLAFp(NMe)K) |
| OM1224 | cyc(R-betaAla-D-f-K(Ahx-Cy5.5)) |
| OM1231 | cyc(FRGDLAFp(NMe)K(Ahx-Cy5.5)) |
| FRX103 | cyc(FRGDLAFp(NMe)K(Ahx-Cy7.5)) |
| FRX109 | cyc(FRADLAFp(NMe)K(Ahx-IRDye ® 800CW)) |
| FRX110 | cyc(FRGDLAFp(NMe)K(Ahx-IRDye ® 800CW)) |

TABLE 1
Selectivity profile of dye-labeled compounds by modification of
unlabeled αvβ6-ligand OM1204. Inactive compound FRX109 for
control experiments by glycine → alanine substitution
( . . . RGD . . . ) → ( . . . RAD . . . )*. IC50-values [nM] (n = 2).

| code | αvβ3 | αvβ5 | αvβ6 | αvβ8 | α5β1 | αIIbβ3 |
|---|---|---|---|---|---|---|
| OM1204 | 632 | >1000 | 0.26 | 23.6 | 72.9 | >1000 |
| OM1231 | n.d. | n.d. | 27 ± 2 | 234 ± 33 | n.d. | n.d. |
| FRX103 | n.d. | n.d. | 45 ± 1 | 481 ± 86 | n.d. | n.d. |
| FRX110 | 408 ± 61 | >10000 | 2.2 ± 0.2 | 129 ± 36 | 37 ± 6 | n.d. |
| FRX109 | >10000 | >10000 | 122 ± 12 | >4000 | >1000 | >1000 |

For the sake of comparison, the above table also includes data on the binding profile of the unmodified parent compound, i.e. compound 18 of WO 2017/046416A (code: OM_1204).

The nature of the dye effect binding affinity of the whole peptide-dye complex to the integrins (Table 1). The compound of the present invention FRX110 exhibits superior binding affinity and selectivity to integrin αvβ6, which qualifies this compound as being particularly suitable for intraoperative imaging.

### 2. Reference Example 1—In Vitro Assessment of Av/36 Expression in Cancer Cells

A molecular biological and functional characterization of invasive carcinoma cells was carried out. The EMT marker protein integrin αvβ6 was identified, which is de novo expressed as part of cellular dedifferentiation and under the influence of signalling molecules (TGF-β) at the mesenchymal border of invasive carcinoma cells. The oncogenic functions and the distinct expression pattern on the proliferation front of invasive carcinoma tissue qualify integrin αvβ6 as a target to detect invasive carcinoma cells and to demarcate them from healthy tissue.

2.1 Immunohistochemical analysis

Immunohistochemical Staining for Integrin αvβ6 was Performed on 4-Pm-Thick Sections from bone-infiltrating FFPE carcinoma samples. Following heat fixation, paraffin slides were deparaffinized and rehydrated. Endogenous peroxidase was blocked with 0.3% (v/v) MeOH/H₂O₂ for 20 min in the dark. After the unmasking of antigen and the blocking of non-specific sites with PBS, 2% (w/v) bovine serum albumin (BSA), slides were incubated with a monoclonal antibody (diluted 1:2.000) directed against human integrin αvβ6 (Biogen Idec, Cambridge, Mass., USA). ⁴⁹ After incubation for 2 h and washing steps in PBS, biotinylated secondary rabbit anti-mouse antibodies (diluted 1:200; Dako, Glostrup, Denmark) were applied, followed by a horseradish peroxidase (HRP)-streptavidin complex (Dako, Glostrup, Denmark) for 30 min. In order to visualize immune complexes, a 0.05% (w/v) solution of diaminobenzidine (Kit 5001; Dako) containing 0.0018% (v/v) H₂O₂ in a 0.05 M Tris-HCl buffer (pH 7.6) was applied to the sections. Brown staining of carcinoma cell membranes indicated positivity for integrin αvβ6. Stainings in the absence of the primary antibodies served as controls and were negative.

2.2 Immunohistochemical Assessment of Integrin αvβ6 Expression

Integrin αvβ6 protein expression was assessed semi-quantitatively by the immunoreactive score (IRS) in different regions of interest (ROIs). Within carcinoma tissue, three ROIs were defined: Invasion front, transition from the invasive front to the centre and the centre of the carcinoma. The locations within carcinoma cell nests, which infiltrated the bone were subdivided in three ROIs: the region bordering stromal tissue, the central area of carcinoma cell nests and cytokeratin deposits within the carcinoma tissue (keratine pearls).

The localisation on the cellular levels was divided into subcellular ROIs: cell membrane, cytosol and the nucleus. Finally, healthy epithelial and stromal tissue was assessed.

For each ROI the IRS criteria were applied: A=percentage of αvβ6 positive cells: 0=no, 1=<10%, 2=10-50%, 3=51-80%, 4=>80% positive cells. B=intensity of staining: 0=no, 1=mild, 2=moderate, 3=intense staining. Final IRS scores were calculated for each ROI by A×B and integrin αvβ6 expression was defined as: 0-1=negative, 2-3=low, 4-8=moderate, 9-12=high.

Differences between the categories were calculated with the paired sample sign test with a significance level of p≤0.001.

FIG. 2 shows the results of this experiment:
A) The Expression of αvβ6 is localized at the invasive front of the infiltrating carcinoma.

In the central area of the carcinoma αvβ6 could not be detected. The surrounding stroma shows inflammatory reactions without induced αvβ6 expression.
B) Integrin αvβ6 expression of infiltrating carcinoma cells in the cancellous bone. Epithelial keratin pearls do not show αvβ6 expression.
C) Illustration of specific αvβ6 expression, which is restricted to the carcinoma cell membranes.

The graph renders the percentage part (y-axis) of the investigated carcinoma specimens (n=55) and illustrates the expression levels in relation to the localisation (x-axis).

In summary, the highest expression levels could be detected in the area of carcinoma invasion. Carcinoma cells infiltrating mesenchymal tissue are characterized by high integrin αvβ6 expression levels.

### 3. Reference Example 2—In Vitro Binding to Cancer Cells

3.1 Cell Lines and Culture Conditions

Human OSCC cell lines HN was purchased from the German Collection of Microorganisms and Cell Cultures, Braunschweig, Germany (DSMZ no. ACC 417 (HN) The HN cell line was established from a cervical lymph node metastasis of an invasive squamous cell carcinoma of the soft palate. The metastasis occurred 7 years after treatment of the primary tumour and metastasized to the lung and brain. ⁴⁹ As control cells, we used the human ovarian cancer cell line OVMZ6 with low integrin avb6 and high integrin αvβ3 expression. ⁵⁰ All cells were cultivated in Dulbecco's Modified Eagle's Medium (DMEM) (Sigma-Aldrich, St. Louis, Mo., USA), supplemented with 10% (v/v) fetal calf serum (FCS) (Gibco, Lifelechnologies™, Carlsbad, Calif., USA).

3.2 Immunocytochemical Detection of Cellular Integrin αvβ6

HN and OVMZ6 cells were grown on fibronectin-coated microchamber slides (Nunc® Lab-Tek® Chamber Slide™ system, Sigma-Aldrich), fixed in 2% (w/v) paraformaldehyde (PFA) for 15 min at room temperature (RT), once washed in PBS and then blocked for 1 h at RT in phosphate-buffered saline (PBS), 2% (w/v) bovine serum albumin (BSA). Monoclonal antibodies directed to integrin αvβ6¹ (1.6 μg/mL) were incubated on the cells in PBS, 1% (w/v) BSA, for 2 h at r.t., followed by the addition a secondary Alexa-488-labeled goat-anti-mouse IgG (0.6 g/mL) for 45 min at r.t. Slides were mounted in PBS and fluorescence intensity evaluated by the Zeiss LSM 700 (Zeiss, Jena, Germany). In order to convert fluorescence staining intensity into colours of a glow scale, the look-up table (LUT) “orange-to-white” provided with the LSM scanning software Zen (Zeiss) was applied: low intensity (red), medium intensity (yellow), and high intensity (white).

The results of this experiment are shown in FIG. 3a. In this figure, the left panel shows integrin αvβ6 expression in HN cells (A,B) and OVMZ6 cells (E,F). HN cells are characterized by high integrin αvβ6 expression levels. In OVMZ6 cells integrin αvβ6 was nearly absent.

The right panel illustrates a low expression of integrin αvβ3 in HN cells (C, D). In contrast, OVMZ6 cells revealed elevated expression levels of integrin αvβ3 (G, I).

3.3 Cell Binding Experiments of Integrin αvβ6-Targeting OM_1231 Conjugate

HN and OVMZ6 cells were cultivated for 24 h on fibronectin-coated microchamber slides at a density of 25×10³/well, thereafter fixed in 2% (w/v) PFA for 15 min at r.t. and washed in PBS. The Cy5.5-conjugated compound OM_1231, dissolved in PBS, 5% (v/v) dimethyl sulfoxide (DMSO), was applied at a final concentration of 10 μM for 1 h at r.t., followed by 3 washes in PBS. In order to prove binding specificity of OM_1231 to integrin αvβ6, its unlabeled analogue OM_1204 was incubated on cells at a 10-fold molar excess for 1 h at r.t. prior to the addition of OM_1231 (10 μM). As control peptide served the non-binding cycl(R^{beta}ADfK) labelled via lysine side-chain NH₂ group with Cy5.5 fluorescent dye (code OM_1224). After the incubation period, cells were washed 6 times in PBS and slides mounted. Fluorescence signal intensity was detected by the microscope Zeiss LSM 700 (Zeiss) as described above. Images were merged by applying for the fluorescence image the LUT “blue to red”: low intensity: blue; medium intensity: green; and high intensity: red.

FIG. 3b shows the results of this experiment:

In FIG. 3b, Panels A-C: HN cells treated with the Cy5.5-labeled compound OM_1231 revealed strong fluorescence signal intensity on cell membranes

D-F: The non-binding Cy5.5-labeled compound (OM_1224) did not result in fluorescence signal intensity.

G-I: Blocking studies: binding competition experiments by adding first unlabeled OM_1204, followed by incubation of cells with its Cy5.5-conjugated analogue (OM_1231) resulted in a strong reduction in fluorescence signal intensity.

J-L: OM_1231 did not recognize cellular integrin αvβ3 on OVMZ6 cells.

M-O: Integrin αvβ6-negative epithelial cells did not reveal any binding of αvβ6 selective RGD-peptides. From ³⁹.

In vitro analyses thus confirm the specific binding of a comparative compound OM_1231, which is a functionalized αvβ6-specific RGD peptide, to αvβ6-positive carcinoma cells.

### 4. Inventive Example 1—In Vivo Binding of Compound of Invention to Cancer Cells

An orthotopic HNSCC PDX (head and neck squamous cell carcinoma patient derived xenotransplant) mouse model was developed that allows the analysis of invasive, orthotropic human carcinoma tissue. HNSCC tissue samples were transplanted into the neck area of NOD/SCID mice. This provides epithelial derived carcinoma tissue in a mesenchymal tissue context to mimic the epithelial-mesenchymal interface between invasive carcinoma tissue and stroma.

In the HNSCC PDX mouse model, integrin αvβ6-specific PET imaging revealed integrin αvβ6 as a sensitive diagnostic marker for invasive carcinoma tissue. ⁴⁵

4.1 Mouse Model with Human Carcinoma Cells

Human carcinoma samples were collected from the proliferative invasive area during surgery and were transplanted into Dulbecco's Modified Eagle Medium (DMEM) containing 1% penicillin/streptomycin and 2.5 μg/ml amphotericin B. The carcinoma tissue was minced into 2×2×2 mm pieces of tissue 1-2 h post excisionem transplanted into the cervical musculature of NOD/SCID mice.

Similarly, healthy oral epithelium was transplanted for control experiments.

The animals are kept in special ventilated cages for keeping mice (Tecniplast IVC). The maximum stocking density in a mouse cage (Type I Superlong, base area 16×37 cm) is based on the weight of the animals in accordance with EU Directive 2010/63. Food (autoclaved mouse food rat/mouse, pelleted 10 mm round, specially treated, housing feed No. 1324SP, Fa. Altromin) and water (acidified drinking water (1N HCl, pH 3.5 Ò 3.0) with a single bottle change per week) received the Animals ad libitum. Special wood granules (Select Fine, Ssniff) served as litter for two changes per week. Nesting material is provided as autoclaved pulp, as well as a red polycarbonate mouse house (Bioscape). The room is specially designed as a livestock room (person-limited access control, care by trained animal care technicians, air conditioning, light-dark rhythm of 12 hours each with twilight phase). The keeping of the animals is carried out in compliance with the conditions set out in EU Directive 2010/63.

Surgical Procedure:

The mouse was fixed and weighed for administration of intraperitoneal anesthesia. Subcutaneous analgesic was administered preoperatively (Rimadyl, 4.0-5.0 mg/kg diluted 1:10, 10 ml/kg body weight), which is also given postoperatively as a pain medication every 24 hours for the first three days postoperatively.

Anesthesia:

For completely antagonizable anesthesia, midazolam (5 mg/kg), medetonidine (0.5 mg/kg) and fentanyl (0.05 mg/kg) are given intraperitoneally with a 27 g cannula. A subsequent dosing took place approximately every 30 minutes with ⅓ of the starting dose. Medication was antagonistic by subcutaneous administration of flumazenil (0.5 mg/kg), atipamezole (2.5 mg/kg) and naloxone (1.2 mg/kg).

Surgical Procedure of Xenogenic Orthotopic Transplantation:

Mice were placed on a warm pad and given Bepanthen Eye Ointment after control of surgical tolerance. The operating area in the area of the cheek and the angle of the jaw was carefully cleaned and the coat was cervically gently shaved on an area of approx. 7×7 mm. The skin was disinfected and the cutis and subcutis were severed with a scalpel. The length of the cut was about 5 mm. The tissue was prepared to the surface of the masseter muscle. The masseter muscle was stumped open and an approx. 3×3×3 mm piece of tissue from a human carcinoma tissue was implanted into the cervical musculature of the mouse. The carcinoma tissue is obtained intraoperatively from the invasion area of a human squamous cell carcinoma, pretreated antiseptically with Braunovidone and promptly provided for xenotransplantation. After implantation of the carcinoma tissue, the surgical site was inspected and closed with Vicryl 6-0 sutures.

Postoperative Management:

Analgesia: The mice were already injected subcutaneously with buprenorphine at the dose of 0.05 mg/kg at the time of anesthesia. Thereafter, buprenorphine is given for 24 hours in an 8 hour rhythm. In addition, the preoperatively prepared Rimadyl is administered every 24 hours.

4.2 Administration of Compound of Invention and Bioimaging

FRX110 was slowly administered as a 10-100 μM solution (200μ1) in sterile PBS via the tail vein of the HNSCC PDX mice and imaging was performed at the given timepoints, as described.

Bioimgaing was performed relying on equipment described in ⁴² using a 750 nm CW laser diode (BWF2-750-0, B & W Tek, Newark, Del., USA) with a maximum power of 300 mW was used to excite the fluorophores. The lighting was carried out by a 250 W halogen lamp (KL-2500 LCD, Schott AG, Mainz, Germany). The laser power was measured at a working distance of 15 cm at 85 mW/cm 2, which is lower than the maximum permissible exposure according to the standard of the American National Standards Institute (ANSI). A short-pass filter (E700SP, Chroma Technology, Rockingham, Vt., USA) was used to eliminate component of the field illumination NIRF signal to preclude interference between the fluorescence detection field and the field illumination light path (F1). Ground glass diffusers (DG10-220, Thorlabs, Newton, N.J., USA) are used to achieve uniform illumination from both light sources (D). The optical signal is resolved by a motorized zoom/focus lens (CVO GAZ11569M, Goyo Optical Inc., Asaka, Saitma, Japan) and spectrally resolved in two channels by a dichroic mirror (700DCXXR, AHF Analysentechnik AG, Tubingen, Germany) (DM). The first channel within the spectrum ranges from 720 to 850 nm, filtered through a NIRF achromatic doublet pair (MAP10100100-B, Thorlabs) (RL1) with an NIRF emission filter (ET810/90, Chroma technology) (F2) and recorded by an iXon electron multiplying charge coupled device (EMCCD, DV897DCS-BV, Andor Technology, Belfast, Northern Ireland). The second channel, which is in the spectral range 450-700 nm, is passed through a visible pair of achromatic doublets (MAP10100100-A, Thorlabs) (RL2) filtered through a short-pass filter (ET700SP-2P, Chroma Technology) (F3) and from a 12-bit color charge coupled device (CCD) camera (pixelfly qe, PCO AG, Kelheim, Germany).

The camera system [based on EMCCD detection (Luca R, Andor Technology). The camera has a fluorescence filter (D850/40m, chroma technology) (F4) and uses the lens (zoom 7000 macro lens, Navitar, N.Y., United States). Camera capture and control was accomplished through Solis software (Solis I, Andor technology) and GPU-based C++ software developed by our group. All data processing implemented in MATLAB (Mathworks Inc., Massachusetts, United States).

4.3 Results

The results of the experiment are shown in FIG. 4a: Bioimaging of proliferative invasive HNSCC tissue in the neck area of a NOD/SCID mouse with FRX110. Imaging was performed from the 1- to the 12^{th} week after xenotransplantation (p. TX) of human HNSCC to image the carcinoma proliferation. The enlarged pictures illustrate bioimaging of proliferating, invasive carcinoma tissue in the 5^{th} week (p. TX) with high tissue to background (TBR) ratios. FIG. 4 reveals specific binding of FRX110 to invasive growing carcinoma xenotransplants, enabling intraoperative molecular bioimaging and demarcation of invasive carcinoma tissue.

4.4 Supplementary Experiment

After exclusion of non-specific signals by FRX110, the molecular bioimaging (=MBI) of integrin αvβ6 was tested with FRX110 in the HNSCC PDX model. The results below are shown in chronological order. In our preliminary studies, an excretion of unbound NIRF tracers was found after 4-6 h without nonspecific accumulation. To set the optimal time with the best ratio between NIRF signal/background signal for an MBI of integrin αvβ6, the time window was set to 24 h p.i. extended. The overview of the test series is shown in Table 2.

TABLE 2

| NOD/ | | | | | | | | | |
|---|---|---|---|---|---|---|---|---|---|
| SCID | Modell | 0.W | 1.W | 4.W | 5.W | 8.W | 10.W | 12.W | FIG. XX |
| 1 | HNSCC- | TX | MBI | MBI | MBI | MBI | MBI | MBI | A1: 1.W, 0-24 h p.i. |
| | PDX | | PET | PET | | | | MBIAS | A2: 1-12.W, 24 h p.i. |
| 2 | HNSCC- | TX | MBI | MBI | MBI | MBI | MBI | MBI | B1: 1.W, 0-24 h |
| | PDX | | PET | PET | | | MBIAS | | B2: 1-12.W, 24 h p.i. |
| 3 | HNSCC- | TX | MBI | MBI | MBI | MBI | MBI | MBI | C1: 1.W, 0-24 h |
| | PDX | | PET | PET | | | | | C2: 1-12.W, 24 h p.i. |
| 4 | HNSCC- | TX | MBI | MBI | MBI | MBI | x | x | D1: 1.W, 0-24 h |
| | PDX | | PET | PET | | MBIAS | | | D2: 1-8.W, 24 h p.i. |
| 5 | HNSCC- | TX | MBI | MBI | MBI | MBI | MBI | MBI | E1: 1.W, 0-24 h |
| | PDX | | PET | PET | | | | | E2: 1-12.W, 24 h p.i. |
| 6 | HNSCC- | TX | MBI | MBI | MBI | MBI | MBI | MBI | F1: 1.W, 0-24 h |
| | PDX | | PET | PET | MBIAS | | | | F2: 1-12.W, 24 h p.i. |

Time of interventions in weeks (W) after transplantation (TX);

post injection (p.i.); Interventions: TX=xenogeneic orthotopic transplantation of human carcinoma tissue; MBI=molecular bioimaging with integrin αvβ6-specific NIRF tracer (FRX110); PET=Positron Emission Tomography 68GA-Avebehexin; MBIAS=molecular bioimaging assisted surgery

After xenotransplantation of human carcinoma tissue, the accumulation and biodistribution of FRX110 in the HNSCC PDX model was first examined after one week (time W1).

For biodistribution analysis, molecular bioimaging (MBI) was performed at 0, 4, 6 and 24 h post injections (p.i.) of the αvβ6-specific NIRF tracer (FRX110) via the tail vein.

After 6 h the tracer revealed renal clearance and was eliminated after 24 h (please see FIG. 5b and corresponding text under Item 5.2 below).

In the following weeks, the MBI of αvβ6 expression in the proliferating carcinoma tissue in the 4th, 5th, 8th, 10th and 12th week post transplantation (p.Tex) at 24 h p. i.

The measurements of the average fluorescence intensity/pixels were made in two regions of interests (ROIs). ROI1 is the localization of xenotransplantation. ROI2 is the reference on the contralateral side, with comparable tissue perfusion.

Each animal is represented in 4 different channels: colored light=clinical situation; Fluorescence=fluorescence signal in black/white, fusion=superposition of colored light and fluorescence channels; in the fluorescence intensity scale the distribution of fluorescence intensity/pixel is shown.

The quotient ROI1/ROI2 of the average fluorescence intensity/pixel is given as tumor tissue to background ratio (TBR). A TBR>1 corresponds to a fluorescence signal above the background signal and by accumulation of FRX110 in the area of carcinoma transplantation (ROI1) relative to ROI2.

The average fluorescence intensity/pixel is shown graphically at each time point. The TBR is tabulated in numerical values as well as graphically over the time course of the test series.

FIG. 4b summarizes the MBI data from the test series shown in Table 2.

The experiments of this example demonstrate that the compound of the present invention selectively accumulates in the tumor cells exhibiting a high fluorescence intensity to yield a high tumor to background ratio.

### 5. Reference Example—Biodistribution

5.1 Biodistribution in the Organism of Comparative Compound

The comparative compound FRX103 was injected i.v. in 10 μmol concentration into HNSCC PDX mice. Relying on fluorescence intensity measurements, the distribution and accumulation of FRX103 in the organism was determined over the time period shown. The results of these experiments are shown in FIG. 5a for a time period of up to 24 h post injection (p.i.).

FRX103 showed a constant accumulation in the area of carcinoma tissue. After 24 h p.i. the ratio between specific signal in the area of the carcinoma tissue and the background signal was best. However, FRX103 does not show any elimination after 24 h but an undesirable residual accumulation in the liver and pancreas.

In control animals undergoing healthy epithelial grafting (control TX) no nonspecific accumulation in the area of transplantation could be observed. Similar to the HNSCC PDX group, however, all animals (n=7) showed a persistent signal in the liver and pancreas after 24 h.

These observations demonstrate that the biodistribution and elimination of the tested FRX103 compound are unsatisfactory.

5.2 Biodistribution in the Organism of Inventive Compound

The above experiment was repeated using the compound FRX110 of the present invention. The results of this experiment are shown in FIG. 5b. It may be derived from this figure that the biodistribution in the timescale 0-24 h post injectionem does not reveal unspecific accumulation or binding (TBR 1.0). Unbound FRX110 is eliminated via the renal system within 24 h. In comparison with FRX103, FRX110 has been demonstrated to be a more suitable compound for bioimaging applications.

5.3 In Vivo Binding of Compound of Invention to Healthy Cells

Control experiments were performed with sham operations and transplantation with healthy control tissue, which were imaged with FRX110 and FRX109 as the control tracer.

Biodistribution was tested in the control TX model and control animals for further investigations.

The specificity of the binding was investigated in the in vivo model with the control tracer FRX109. FRX109 was visualized in mice with a sham graft (sham TX) of human carcinoma tissue (no tissue was transplanted). FRX110 was visualized in an unoperated control mouse, a sham TX mouse, and in a mouse that had undergone transplantation of human, healthy epithelium (control TX).

Table 3 gives an overview of the control experiments in order to be able to exclude nonspecific binding of the NIRF tracer.

TABLE 3

| | FRX109 | FRX110 | | |
|---|---|---|---|---|
| | (Controltracer) | (NIRF-Tracer) | controle of | controls |
| Sham-TX | | × | unspecific enrichment | A |
| | | | after intervention | |
| Sham-TX | × | | unspecific enrichment | B |
| | | | of IRDye800CW | |
| control-TX | | × | non-specific accumulation | C |
| | | | in the epithelium or due | |
| | | | to reactive changes after | |
| | | | transplantation of | |
| | | | xenogeneic tissue | |
| control-TX | × | | nonspecific accumulation | D |
| | | | of the NIRF tracer in the | |
| | | | epithelium or due to | |
| | | | reactive changes after | |
| | | | transplantation of | |
| | | | xenogeneic tissue | |
| Control mouse | | × | Biodistribution of the | E |
| | | | NIRF tracer without | |
| | | | operative intervention | |

FIG. 5c shows that FRX110 and FRX109 revealed good renal clearance 6 h after injection (p.i.) without unspecific tracer enhancement and low tissue to background (TBR) ratios.

### 6. Inventive Example 2—In Vivo Use of Compound of Invention in Intraoperative Detection of Cancer Cells

In the HNSCC PDX mouse model, intraoperative molecular bioimaging-assisted carcinoma resections were performed. FIG. 6 shows histological/immunohistological representations of the excised carcinomas. Intraoperative bioimaging according to the present invention provided information on the localization, extent, and tumour biologic characteristics of malignant tissue becomes available intraoperatively in real time.

It was further demonstrated in the preclinical HNSCC PDX mouse model that bioimaging-assisted carcinoma resection allows the intraoperative detection of invasive carcinoma tissue and the control of resection status.

### REFERENCES

1 Binahmed, A., Nason, R. W. & Abdoh, A. A. The clinical significance of the positive surgical margin in oral cancer. Oral Oncol 43, 780-784, doi:S1368-8375(06)00280-6 [pii] 10.1016/j.oraloncology.2006.10.001 (2007).
2 Loree, T. R. & Strong, E. W. Significance of positive margins in oral cavity squamous carcinoma. Am J Surg 160, 410-414 (1990).
3 Hinni, M. L. et al. Surgical margins in head and neck cancer: A contemporary review. Head Neck, doi:10.1002/hed.23110 (2012).
4 Jesse, R. H. & Sugarbaker, E. V. Squamous cell carcinoma of the oropharynx: why we fail. Am J Surg 132, 435-438 (1976).
5 Nieberler, M. et al. Evaluation of intraoperative cytological assessment of bone resection margins in patients with oral squamous cell carcinoma. Cancer cytopathology 122, 646-656, doi:10.1002/cncy.21428 (2014).
6 Nieberler, M. et al. Clinical Impact of Intraoperative Cytological Assessment of Bone Resection Margins in Patients with Head and Neck Carcinoma. Annals of surgical oncology, doi:10.1245/s10434-016-5208-1 (2016).
7 Nieberler, M. et al. Intraoperative cell isolation for a cytological assessment of bone resection margins in patients with head and neck cancer. Br J Oral Maxillofac Surg, doi:10.1016/j.bjoms.2017.02.006 (2017).
8 Koch, M. & Ntziachristos, V. Advancing Surgical Vision with Fluorescence Imaging. Annu Rev Med 67, 153-164, doi:10.1146/annurev-med-051914-022043 (2016).
9 Mery, E. et al. Fluorescence-guided surgery for cancer patients: a proof of concept study on human xenografts in mice and spontaneous tumors in pets. Oncotarget 8, 109559-109574, doi:10.18632/oncotarget.22728 (2017).
10 Mamuya, F. A. & Duncan, M. K. aV integrins and TGF-beta-induced EMT: a circle of regulation. Journal of cellular and molecular medicine 16, 445-455, doi:10.1111/j.1582-4934.2011.01419.x (2012).
11 Breuss, J. M., Gillett, N., Lu, L., Sheppard, D. & Pytela, R. Restricted distribution of integrin beta 6 mRNA in primate epithelial tissues. The journal of histochemistry and cytochemistry: official journal of the Histochemistry Society 41, 1521-1527 (1993).
12 Niu, G. & Chen, X. Why integrin as a primary target for imaging and therapy. Theranostics 1, 30-47 (2011).
13 Breuss, J. M. et al. Expression of the beta 6 integrin subunit in development, neoplasia and tissue repair suggests a role in epithelial remodeling. J Cell Sci 108 (Pt 6), 2241-2251 (1995).
14 Bates, R. C. et al. Transcriptional activation of integrin beta6 during the epithelial-mesenchymal transition defines a novel prognostic indicator of aggressive colon carcinoma. J Clin Invest 115, 339-347, doi:10.1172/JC123183 (2005).
15 Janes, S. M. & Watt, F. M. Switch from alphavbeta5 to alphavbeta6 integrin expression protects squamous cell carcinomas from anoikis. The Journal of cell biology 166, 419-431, doi:10.1083/jcb.200312074 (2004).
16 Jiang, X. et al. Switch from alphavbeta5 to alphavbeta6 integrin is required for CD9-regulated keratinocyte migration and MMP-9 activation. FEBS letters 588, 4044-4052, doi:10.1016/j.febslet.2014.09.027 (2014).
17 Guo, W. & Giancotti, F. G. Integrin signalling during tumour progression. Nature reviews. Molecular cell biology 5, 816-826, doi:10.1038/nrm1490 (2004).
18 Balzac, F. et al. E-cadherin endocytosis regulates the activity of Rap1: a traffic light GTPase at the crossroads between cadherin and integrin function. J Cell Sci 118, 4765-4783, doi:10.1242/jcs.02584 (2005).
19 Oloumi, A., McPhee, T. & Dedhar, S. Regulation of E-cadherin expression and beta-catenin/Tcf transcriptional activity by the integrin-linked kinase. Biochimica et biophysica acta 1691, 1-15, doi:10.1016/j.bbamcr.2003.12.002 (2004).
20 Bandyopadhyay, A. & Raghavan, S. Defining the role of integrin alphavbeta6 in cancer. Current drug targets 10, 645-652 (2009).
21 Kawashima, A. et al. Expression of alphav integrin family in gastric carcinomas: increased alphavbeta6 is associated with lymph node metastasis. Pathol Res Pract 199, 57-64 (2003).
22 Ahmed, N. et al. Overexpression of alpha(v)beta6 integrin in serous epithelial ovarian cancer regulates extracellular matrix degradation via the plasminogen activation cascade. Carcinogenesis 23, 237-244 (2002).
23 Ramos, D. M. et al. Expression of integrin beta 6 enhances invasive behavior in oral squamous cell carcinoma. Matrix biology: journal of the International Society for Matrix Biology 21, 297-307 (2002).
24 Zhang, Z. Y. et al. Integrin alphanvbeta6 acts as a prognostic indicator in gastric carcinoma. Clin Oncol (R Coll Radiol) 20, 61-66, doi:10.1016/j.clon.2007.09.008 (2008).
25 Hayashido, Y. et al. Overexpression of integrin alphav facilitates proliferation and invasion of oral squamous cell carcinoma cells via MEK/ERK signaling pathway that is activated by interaction of integrin alphavbeta8 with type collagen. Int J Oncol 45, 1875-1882, doi:10.3892/ijo.2014.2642 (2014).
26 Dong, X. et al. Force interacts with macromolecular structure in activation of TGF-beta. Nature 542, 55-59, doi:10.1038/nature21035 (2017).
27 Ramos, D. M., Dang, D. & Sadler, S. The role of the integrin alpha v beta6 in regulating the epithelial to mesenchymal transition in oral cancer. Anticancer Res 29, 125-130 (2009).
28 Yang, G. Y. et al. Integrin alpha v beta 6 mediates the potential for colon cancer cells to colonize in and metastasize to the liver. Cancer science 99, 879-887, doi:10.1111/j.1349-7006.2008.00762.x (2008).
29 Munshi, H. G. & Stack, M. S. Reciprocal interactions between adhesion receptor signaling and MMP regulation. Cancer Metastasis Rev 25, 45-56, doi:10.1007/s10555-006-7888-7 (2006).
30 Saldanha, R. G. et al. Proteomic identification of lynchpin urokinase plasminogen activator receptor protein interactions associated with epithelial cancer malignancy. J Proteome Res 6, 1016-1028, doi:10.1021/pr060518n (2007).
31 Li, X. et al. Alphavbeta6-Fyn signaling promotes oral cancer progression. J Biol Chem 278, 41646-41653, doi:10.1074/jbc.M306274200 (2003).
32 Hecht, J. L., Dolinski, B. M., Gardner, H. A., Violette, S. M. & Weinreb, P. H. Overexpression of the alphavbeta6 integrin in endometrial cancer. Appl Immunohistochem Mol Morphol 16, 543-547, doi:10.1097/PAI.0b013e31816bc5ee (2008).
33 Al-Hazmi, N., Thomas, G. J., Speight, P. M. & Whawell, S. A. The 120 kDa cell-binding fragment of fibronectin up-regulates migration of alphavbeta6-expressing cells by increasing matrix metalloproteinase-2 and -9 secretion. European journal of oral sciences 115, 454-458, doi:10.1111/j.1600-0722.2007.00481.x (2007).
34 Impola, U. et al. Differential expression of matrilysin-1 (MMP-7), 92 kD gelatinase (MMP-9), and metalloelastase (MMP-12) in oral verrucous and squamous cell cancer. J Pathol 202, 14-22, doi:10.1002/path.1479 (2004).
35 Thomas, G. J., Hart, I. R., Speight, P. M. & Marshall, J. F. Binding of TGF-beta1 latency-associated peptide (LAP) to alpha(v)beta6 integrin modulates behaviour of squamous carcinoma cells. Br J Cancer 87, 859-867, doi:10.1038/sj.bjc.6600545 (2002).
36 Thomas, G. J., Lewis, M. P., Hart, I. R., Marshall, J. F. & Speight, P. M. AlphaVbeta6 integrin promotes invasion of squamous carcinoma cells through up-regulation of matrix metalloproteinase-9. Int J Cancer 92, 641-650 (2001).
37 Niu, J. et al. Integrin expression in colon cancer cells is regulated by the cytoplasmic domain of the beta6 integrin subunit. Int J Cancer 99, 529-537, doi:10.1002/ijc.10397 (2002).
38 Nieberler, M. et al. Exploring the Role of RGD-Recognizing Integrins in Cancer. Cancers (Basel) 9, doi:10.3390/cancers9090116 (2017).
39 Maltsev, O. V. et al. Stable Peptides Instead of Stapled Peptides: Highly Potent alphavbeta6-Selective Integrin Ligands. Angew Chem Int Ed Engl 55, 1535-1539, doi:10.1002/anie.201508709 (2016).
40 WO 2017/046416 A and references cited therein.
40a T. G. Kapp, F. Rechenmacher, S. Neubauer, O. V. Maltsev, A. E. Cavalcanti-Adam, R. Zarka, U. Reuning, J. Notni, H.-J. Wester, C. Mas-Moruno, J. P. Spatz, B. Geiger, H. Kessler; A Comprehensive Evaluation of the Activity and Selectivity Profile of Ligands for RGD-binding Integrins. Scientific Reports 2017 7:39805 | DOI: 10.1038/srep39805.
40b Tummers W. S., Kimura R. H., Abou-Elkacem L., Vahrmeijer A. L., Swijnenburg R. J., Willmann J. K., Gambhir S. S. Development and preclinical validation of a cysteine knottin peptide targeting Integrin αvβ6 for near-infrared fluorescent-guided surgery in pancreatic cancer. Clin Cancer Res. 2018 Jan. 3. pii: clincanres.2491.2017. doi: 10.1158/1078-0432.CCR-17-2491. [Epub ahead of print]
41 Koch M., Ntziachristos V. Advancing Surgical Vision with Fluorescence Imaging. Annu Rev Med. 2016; 67:153-64. doi: 10.1146/annurev-med-051914-022043.
42 Gorpas D., Koch M., Anastasopoulou M., Klemm U., Ntziachristos V. Benchmarking of fluorescence cameras through the use of a composite phantom. J Biomed Opt. 2017 Jan. 1; 22(1):16009. doi: 10.1117/1.JBO.22.1.016009.
43 Rosenthal E. L., Warram J. M., de Boer E., et al. Safety and tumor-specificity of cetuximab-IRDye800 for surgical navigation in head and neck cancer. Clin. Cancer Res. 2015.21: 3658-66
44 van Scheltinga A. G. T., van Dam G. M., Nagengast W. B., et al. Intraoperative near-infrared fluorescence tumor imaging with vascular endothelial growth factor and human epidermal growth factor receptor 2 targeting antibodies. J. Nucl. Med. 2011.52:1778-85
45 Notni, J. et al. In Vivo PET Imaging of the Cancer Integrin alphavbeta6 Using 68Ga-Labeled Cyclic RGD Nonapeptides. Journal of nuclear medicine: official publication, Society of Nuclear Medicine 58, 671-677, doi:10.2967/jnumed.116.182824 (2017).
46 Fields, G. B. & Noble, R. L. Solid phase peptide synthesis utilizing 9-fluorenylmethoxycarbonyl amino acids. International journal of peptide and protein research 35, 161-214 (1990).
47 Chatterjee, J., Gilon, C., Hoffman, A. & Kessler, H. N-methylation of peptides: a new perspective in medicinal chemistry. Accounts of chemical research 41, 1331-1342, doi:10.1021/ar8000603 (2008).
48 Frank, A. O. et al. Conformational control of integrin-subtype selectivity in isoDGR peptide motifs: a biological switch. Angewandte Chemie (International ed. in English)
49, 9278-9281, doi:10.1002/anie.201004363 (2010).
49 H. Kawamata, K. Nakashiro, D. Uchida, K. Harada, H. Yoshida, M. Sato, Int. J. Cancer 1997, 70, 120-127.
50 M. A. Muller, J. Opfer, L. Brunie, L. A. Volkhardt, E. K. Sinner, D. Boettiger, A. Bochen, H. Kessler, K. E. Gottschalk, U. Reuning, J. Mol. Biol. 2013, 425, 2988-3006.

1. A compound suitable for intraoperative imaging, which is characterized by the general formula (I):
A-(L)ₙ-B (I)
wherein A represents a moiety, which is derived from cyc(FRGDLAFp(NMe)K), L represents a linker, n is 0 or 1 and B represents a moiety derived from a fluorescent dye, which is a moiety having the following structure: 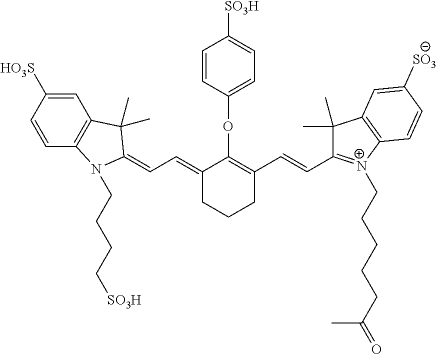
wherein the dye moiety B is bonded to the linker in case of n=1 or to moiety A in case of n=0 via the carbon atom of the carbonyl group, or pharmaceutically acceptable salts thereof.

2. A compound, or pharmaceutically acceptable salt thereof, suitable for intraoperative imaging according to claim 1, wherein the linker is derived from an ω-amino alkyl carboxylic acid with 5 to 7 carbon atoms.

3. A compound, or pharmaceutically acceptable salt thereof, suitable for intraoperative imaging according to claim 1, wherein the linker is bonded to the ω-amino group of the lysine residue of moiety A.

4. A compound, or pharmaceutically acceptable salt thereof, suitable for intraoperative imaging according to claim 1, wherein the compound has the following structure:

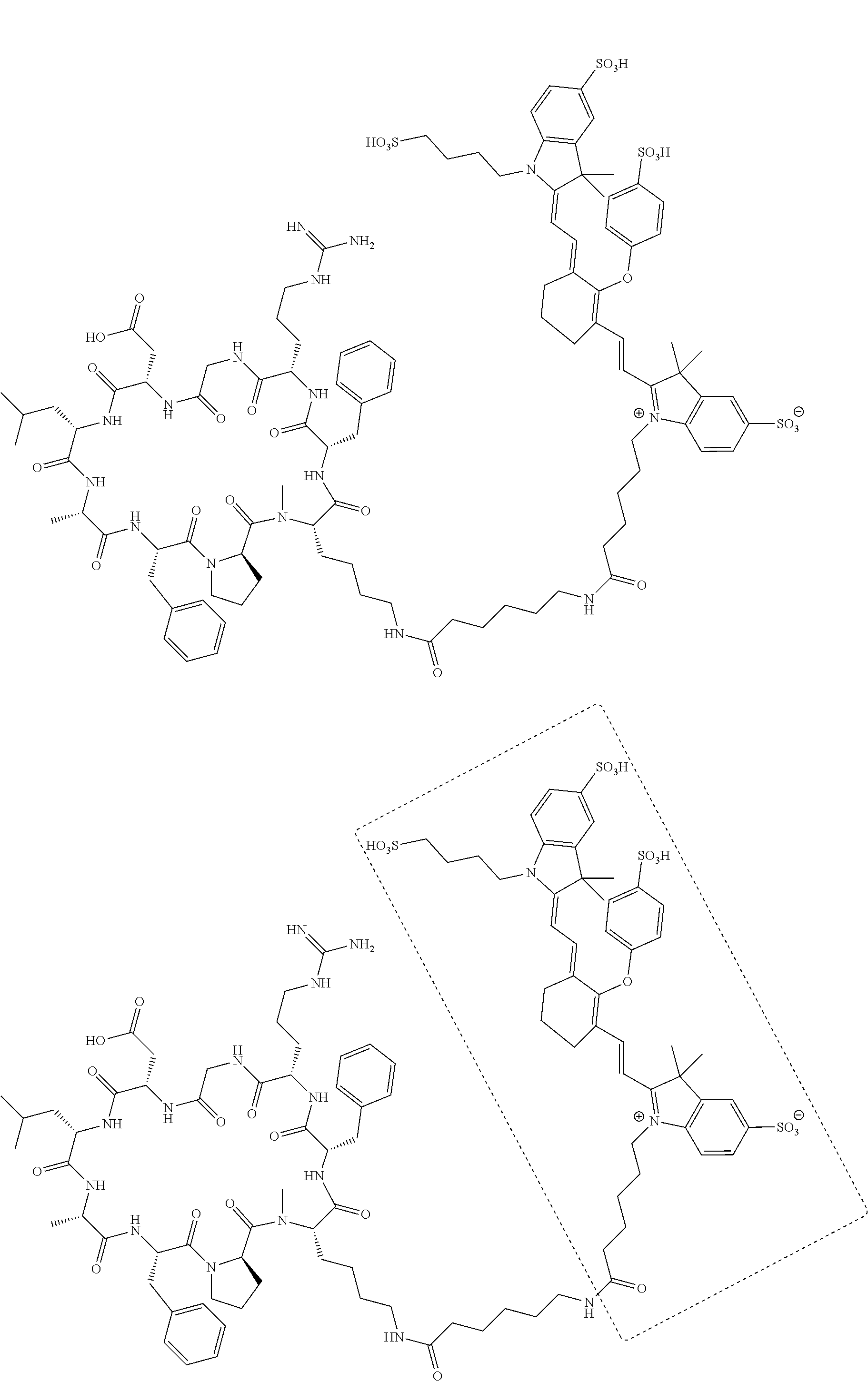

5. A method of manufacturing the compound, or pharmaceutically acceptable salt thereof, according to claim 1, which comprises the steps of
(i) providing a linker that carries moiety A;
(ii) providing a fluorescent dye containing the moiety B;
(iii) reacting the fluorescent dye with the moiety A-carrying linker such that a covalent bond is formed between the linker and moiety B derived from the fluorescent dye; or
(i′) providing a linker that carries a moiety B derived from a fluorescent dye as specified in claim 1;
(ii′) providing a compound that contains moiety A;
(iii′) reacting the compound containing moiety A with the moiety-carrying linker such that a covalent bond is formed between the linker and the moiety A.

6. A method of interoperative surgery for the treatment of cancer in a patient, comprising administration to the patient of a compound of claim 1, or pharmaceutically acceptable salt thereof.

7. The method according to claim 6, wherein the method comprises the steps of
(a) systemically administering the compound, or pharmaceutically acceptable salt thereof, according to claim 1 to a patient;
(b) Irradiating the tissue with suspected tumor cells with light capable of inducing fluorescence emission by the dye moiety.
(c) Detecting fluorescence emission.
(d) Surgically removing tissue that exhibits fluorescence emission or that is surrounded by tissue exhibiting fluorescence emission.

8. The method of claim 6, wherein the cancer is selected from colon cancer, gastric carcinoma, oral squamous cell carcinoma (OSCC), pancreatic ductal adenocarcinoma, intestinal adenocarcinoma, head and neck squamous cell carcinoma (HNSCC), invasive endometrial carcinoma, basal cell carcinoma, breast cancer, endometrial cancer, gastric cancer, liver cancer, non small cell lung cancer, lung cancer brain metastases, ovarian cancer, pancreatic cancer and prostate cancer.

9. The compound of claim 2, or pharmaceutically acceptable salt thereof, wherein the linker derived from an ω-amino alkyl carboxylic acid with 5 to 7 carbon atoms is 6-aminohexanoic acid.

10. The compound of claim 9, or pharmaceutically acceptable salt thereof, wherein the linker is bonded to the ω-amino group of the lysine residue of moiety A.
